(19) United States
(12) Patent Application Publication
(10) Pub. No.: US 2020/0205846 A1
(43) Pub. Date: Jul. 2, 2020

(54) SURGICAL TOOLS

(71) Applicant: MEDROBOTICS CORPORATION, Raynham, MA (US)

(72) Inventors: David Baker, Cumberland, RI (US); Michael S. Castro, Raynham, MA (US); Richard S. Walsh, Bethany Beach, DE (US); R. Maxwell Flaherty, Topsfield, MA (US); J. Christopher Flaherty, Auburndale, FL (US)

(21) Appl. No.: 16/631,283

(22) PCT Filed: Jul. 17, 2018
(86) PCT No.: PCT/US18/42449

§ 371 Date: Jan. 15, 2020

Related U.S. Application Data
(60) Provisional application No. 62/533,644, filed on Jul. 17, 2017.
(60) Provisional application No. 62/582,283, filed on Nov. 6, 2017.
(60) Provisional application No. 62/614,263, filed on Jan. 5, 2018.
(60) Provisional application No. 62/614,346, filed on Jan. 5, 2018.
(60) Provisional application No. 62/613,899, filed on Jan. 5, 2018.
(60) Provisional application No. 62/614,223, filed on Jan. 5, 2018.
(60) Provisional application No. 62/614,224, filed on Jan. 5, 2018.
(60) Provisional application No. 62/614,228, filed on Jan. 5, 2018.
(60) Provisional application No. 62/614,225, filed on Jan. 5, 2018.
(60) Provisional application No. 62/614,240, filed on Jan. 5, 2018.
(60) Provisional application No. 62/614,235, filed on Jan. 5, 2018.

(51) Int. Cl. A61B 17/295; A61B 17/29

(52) U.S. Cl. CPC A61B 17/295; A61B 2017/2902; A61B 17/2909

(57) ABSTRACT

A surgical instrument comprises a proximal end. A distal end has an articulation region, a movement of which is controlled from the proximal end. A shaft is between the proximal end and the distal end. A plurality of steering cables extend from the proximal end to the distal end. An end effector comprises a clevis, the clevis including at least one channel that receives a portion of a steering cable of the plurality of steering cables.

### RELATED APPLICATIONS

This application claims the benefit of U.S. Provisional Application No. 62/533,644, filed Jul. 17, 2017, the content of which is incorporated herein by reference in its entirety.

This application claims the benefit of U.S. Provisional Application No. 62/614,263, filed Jan. 5, 2018, the content of which is incorporated herein by reference in its entirety.

This application claims the benefit of U.S. Provisional Application No. 62/582,283, filed Nov. 6, 2017, the content of which is incorporated herein by reference in its entirety.

This application claims the benefit of U.S. Provisional Application No. 62/614,346, filed Jan. 5, 2018, the content of which is incorporated herein by reference in its entirety.

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18, 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, the content of which is incorporated herein by reference in its entirety.

This application is related to U.S. Design Application No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### FIELD

The present inventive concepts generally relate to the field of surgical tools, and more particularly, to articulating surgical tools and tool sheaths, methods of deploying articulating surgical tools and tool sheaths, and methods of forming the same.

### BACKGROUND

As less invasive medical techniques and procedures become more widespread, medical professionals, such as surgeons, may require articulating surgical tools to perform such less invasive medical techniques and procedures from outside the human body. However, conventional articulating surgical tools, such as endoscopes and other types of tools, may have limited turning radii and reduced payload stability at high articulation ranges.

### SUMMARY

In an aspect, the present inventive concepts are directed to a surgical instrument. The surgical instrument comprises a proximal end; a distal end having an articulation region, a movement of which is controlled from the proximal end; and a shaft between the proximal end and the distal end; a plurality of steering cables extending from the proximal end to the distal end; and an end effector comprising a clevis, the clevis including at least one channel that receives a portion of a steering cable of the plurality of steering cables.

In an embodiment, the at least one channel comprises a c-shaped channel.

In another embodiment, the at least one channel comprises: a first end at a proximal surface of the clevis; a second end at a proximal surface of the clevis, spaced apart from the first end; and a middle region between the first and second ends.

In another embodiment, the at least one channel includes a pinch region at least one of the first end, the second end or the middle region for securing a portion of a steering cable in the clevis.

In another embodiment, the at least one channel is positioned at a side portion of the clevis.

In another embodiment, a mid-region of the steering cable is positioned in the channel, the steering cable being fixed in the channel.

In another embodiment, the clevis includes a first channel for receiving a mid-portion of a first steering cable of the plurality of steering cables and a second channel for receiving a mid-portion of a second steering cable of the plurality of steering cables.

In another embodiment, each of the first and second steering cables includes a first portion at one side of the mid-portion that extends through first holes in segment links of the articulation region and also through a length of the shaft to the proximal end and a second portion at another side of the mid-portion that extends through second holes in the segment links and also through the length of the shaft to the proximal end.

In another embodiment, the surgical instrument further comprises a ring positioned about the proximal end of the clevis over the first and second channels.

In another embodiment, the clevis includes a reduced-width neck region having a width or diameter less than a proximal end of the clevis, the ring encompassing the first and second channels and the mid-portions of the first and second steering cables in the neck region of the clevis.

In another embodiment, the proximal end includes a handle, the handle including a plurality of controls for controlling a movement of the articulation region.

In another embodiment, the controls of the handle control an advancement and retraction of the steering cables, which in turn articulate the articulation region.

In another embodiment, the end effector includes one or more tools, including at least one of a claw, a pair of scissors, a cutter, a knife, an ablator, a cauterizer, a drug delivery apparatus, a radiation source such as a light-delivery element, an energy delivery element such as an RF or EKG electrode, a sensor such as a pressure sensor, blood sensor, a camera, a magnet, a heating element, or a cryogenic element.

In another embodiment, the end effector includes at least one of a grasper, a light-delivery element, a dual-bladed cutting tool, a single bladed cutting tool, or a forceps.

In another embodiment, the articulation region includes a plurality of articulating links constructed and arranged to articulate relative to each other based on forces applied to the steering cables.

In another embodiment, the clevis is constructed and arranged to articulate relative to a distal articulating link of the plurality of articulating links.

In another embodiment, the clevis comprises a concave region that mates with a corresponding convex region of the distal articulating link.

In another embodiment, the clevis comprises a convex region that mates with a corresponding concave region of the distal articulating link.

In another embodiment, the proximal end comprises a handle that applies the forces to the steering cables.

In another embodiment, the clevis further comprises a hinge at a distal end of the clevis, about which at least one element of the end effector pivots.

In another embodiment, the end effector includes two elongated elements that intersect at the hinge point, and that open and close by at least one of the two elongated elements rotating about the hinge relative to the other.

In another embodiment, the end effector includes a space between the two elongated elements when distal ends of the elongated elements directly abut each other.

In another embodiment, a width or diameter of the articulation region is greater than a width or diameter of the clevis.

In another embodiment, the end effector comprises a light-delivering element and wherein a proximal end of the light delivering element extends from the proximal end of the surgical instrument.

In another embodiment, the surgical instrument further comprises a connector coupled to the proximal end of the light-delivering element.

In another embodiment, the connector includes a Tuohy Borst valve for retaining the light-delivering element.

In another embodiment, the proximal end of the surgical instrument includes a steering mechanism including an articulation limiting element that limits a motion of the surgical instrument in order to prevent breakage of an optical fiber of the light-delivering element.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1A illustrates a side perspective view of one embodiment of a surgical instrument, consistent with the present inventive concepts.

FIG. 1B illustrates a perspective, cutaway view of another embodiment of a surgical instrument, consistent with the present inventive concepts.

FIG. 1C illustrates a side perspective view of another embodiment of a surgical instrument, consistent with the present inventive concepts.

FIGS. 2A-E illustrate perspective views of various embodiments of an end effector for a surgical instrument, consistent with the present inventive concepts.

FIGS. 3A-C illustrate side views of a grasper end effector, in a closed and open position, respectively, for a surgical instrument, consistent with the present inventive concepts.

FIG. 4 illustrates a side perspective view of a surgical instrument with a light-delivering end effector, consistent with the present invention.

FIGS. 4A-C illustrate various views of portions of a handle of the surgical instrument of FIG. 4, consistent with the present inventive concepts.

FIGS. 5A-C illustrate side views of a scissors end effector in a closed position and in an open position, respectively, consistent with the present inventive concepts.

FIGS. 6A-B illustrate a partial cutaway side view of a knife-based end effector in a retracted position and a side perspective view of the end effector in an extended position, respectively, consistent with the present inventive concepts.

FIGS. 7A-C illustrate side views of a forceps-based end effector in a closed position and a side perspective view of the end effector in an open position, respectively, consistent with the present inventive concepts.

FIG. 8A illustrates a perspective view of an embodiment of the clevis and the distal ring, consistent with the present inventive concepts. FIG. 8B illustrates a perspective exploded view of an embodiment of the clevis, the end effector, and an actuating cable, consistent with the present inventive concepts. FIG. 8C illustrates a side view of an embodiment of the clevis, steering cable, articulating links, and distal ring, consistent with the present inventive concepts.

FIGS. 9A-C, illustrates side views of an adaptor and two detachable end effectors, respectively, consistent with the present inventive concepts.

### DETAILED DESCRIPTION

Reference will now be made in detail to the present embodiments of the technology, examples of which are illustrated in the accompanying drawings. The same reference numbers are used throughout the drawings to refer to the same or like parts.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a laser, a light emitting diode, and/or a light bulb), sound (e.g. a transducer comprising a piezo crystal configured to deliver ultrasound energy), pressure, heat energy, cryogenic energy, chemical energy, mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Provided herein are surgical tools with enhanced features. In some embodiments, the end effector is reduced in width, allowing for a narrower end effector profile and improved surgical clearance. In some embodiments, the tool body is reduced in width, allowing for improved maneuverability. In some embodiments, the surgical tools include an articulation region at a distal end thereof, the articulation region including a plurality of links or segments that articulate relative to each other. In some embodiments, a clevis is positioned between the articulation region and the end effector, the clevis supporting the end effector. In some embodiments, the clevis includes an articulation feature that allows for articulation, effectively causing the clevis to operate as a distal link of the articulation region. In some embodiments, the articulation feature is a concave region that mates with a corresponding convex region of a neighboring link. In some embodiments, the articulation feature is a convex region that mates with a corresponding concave region of a neighboring link. In some embodiments, the clevis comprises a C-shaped articulation cable channel. The C-shaped channel provides a terminus for an articulation cable, allowing a single cable to operate as first and second articulation cables for the surgical tool.

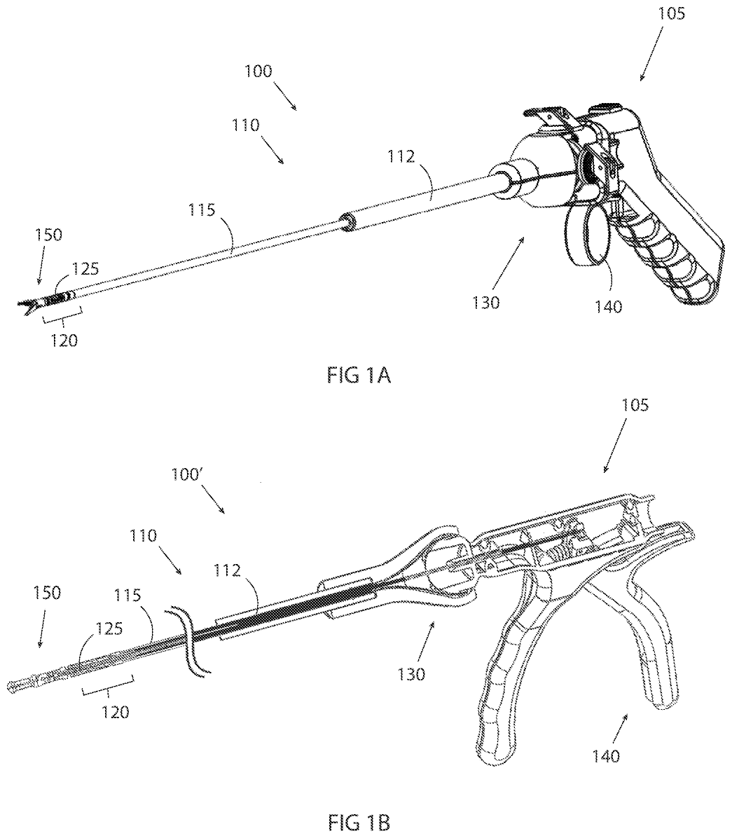

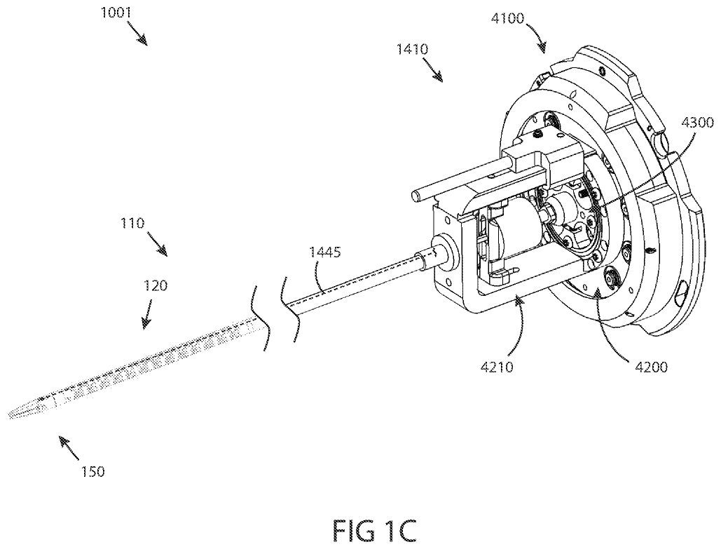

Referring now to FIG. 1A, a side perspective view, of one embodiment of a surgical instrument is illustrated, consistent with the present inventive concepts. Surgical instrument 100, also referred to as a surgical tool, can be a part of a system for performing a medical procedure, such as a system including a robotic probe (e.g. a robotically controlled introducer) and/or a robotically controlled tool. For example, surgical instrument 100 can be provided with an articulating robotic probe, for example, when instrument 100 is slidably positioned within a working channel of the probe and/or a side port or guide hole of an articulating probe, such as the probe described in applicant's co-pending U.S. patent application Ser. No. 14/402,224, filed Nov. 19, 2014, the contents of which are incorporated herein by reference for all purposes. An operator such as a surgeon or other medical professional, can manipulate or otherwise control the functions and movement of instrument 100.

In some embodiments, surgical instrument 100 comprises an articulating surgical instrument in which at least a portion (e.g. a distal portion) can be controllably (e.g. manually and/or robotically) articulated. For example, surgical instrument 100 can include a handle 105, a steering mechanism 130, and an articulation region 120 as shown. Steering mechanism 130 and other components of surgical instrument 100 can be of similar construction and arrangement to the similar components described in: applicant's co-pending U.S. patent application Ser. No. 14/402,224, filed Nov. 19, 2014; applicant's issued U.S. Pat. No. 9,517,059, filed Nov. 2, 2015; and applicant's co-pending U.S. patent application Ser. No. 15/350,549, filed Nov. 14, 2016; the contents of each of which are incorporated herein by reference in their entirety, for all purposes. Surgical instrument 100 can also include a surgical tool shaft, shaft 110, comprising a rigid portion 112 and a flexible portion 115. A distal end of shaft rigid portion 112 can be coupled to a proximal end of shaft flexible portion 115. A distal end of shaft 110 can be directly or indirectly coupled to articulation region 120. Shaft rigid portion 112 and shaft 110 can extend along a same axial direction as articulation region 120, or a different direction, for example, during movement of articulation region 120. Articulation region 120 can comprise multiple articulating links, links 125. In some embodiments, at least a portion of shaft 110 and or articulation region 120 comprises a diameter of less than 5 mm, such as less than 4 mm, less than 3 mm, or less than 2 mm.

Handle 105 is at a proximal end of surgical instrument 100, and can be constructed and arranged to control one or more movements of surgical instrument 100. Handle 105 can be constructed and arranged to include a palm-held grip, a scissors handle, a thumb/index/middle finger grip, a pistol grip, or a reciprocating trigger. Handle 105 can include a plurality of controls that control movement of surgical instrument 100, for example, controlling one or more of: the steering of articulation region 120, the rotation, articulation, and/or activation of an end effector 150 positioned at a distal end of surgical instrument 100, and so on. For example, one of the controls of handle 105 can include an articulation mechanism 140 that serves as a ratcheting trigger with respect to the end effector 150. Activation of the loop-style ratcheting trigger (e.g. a user pulls the loop-style ratcheting trigger with one or two fingers) induces a movement of end effector 150.

In some embodiments, surgical instrument 100 can be robotically controlled, such as described in U.S. Provisional Application entitled “Robotically Controlled Surgical Tool”, by Mitchell, et al., filed of even date herewith and the content of which is incorporated herein by reference.

A movement of handle 105 can provide tension or slack to one or more steering cables 170 (see FIGS. 1B, 3A, and 5A), thereby adjusting an articulation state of articulation region 120. An articulation of handle 105 configured with a single degree of freedom can cause articulation region 120 to move in a single plane along a single pathway of motion. An articulation of handle 105 configured with two degrees of freedom can permit articulation region 120 to be manipulated to reach anywhere on a surface of at least a partial sphere. Steering mechanism 130 can include a universal joint type interface between shaft 110 and handle 105, as described in applicant's issued U.S. Pat. No. 9,517,059, filed Nov. 2, 2015, the contents of which are incorporated herein by reference for all purposes.

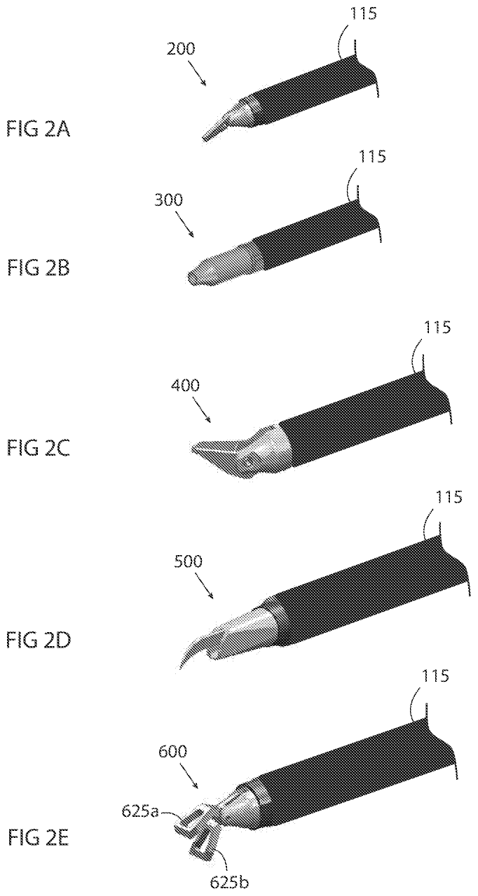

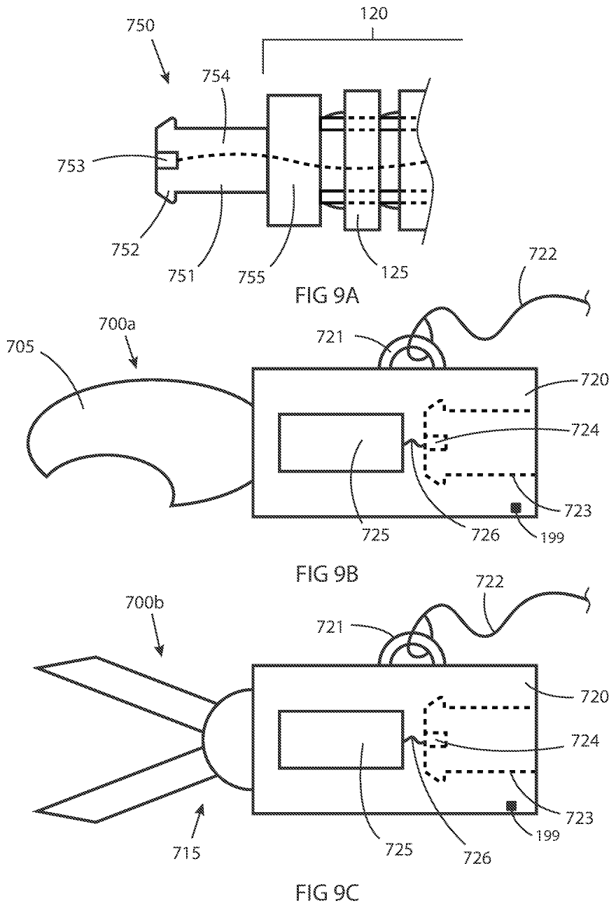

The end effector 150 at the distal end of surgical instrument 100 can include one or more tools, as described herein in reference to FIGS. 2-9. A tool can include but not be limited to a claw, scissors, a cutter, a knife, an ablator, a cauterizer, a drug delivery apparatus, a radiation source such as a light-delivery element, an energy delivery element such as an RF or EKG electrode, a sensor such as a pressure sensor or a blood sensor, a camera, a magnet, a heating element, a cryogenic element, or a combination thereof. In some embodiments, the end effector 150 is constructed and arranged to articulate between 0° and 180°, or more, with respect to an axis of extension of shaft 110. In some embodiments, end effector 150 can comprise an adaptor and a removable tool, as described in reference to FIGS. 9A-C herebelow.

Referring now to FIG. 1B, a side perspective cutaway view of another embodiment of a surgical instrument is illustrated, consistent with the present inventive concepts. Surgical instrument 100′, also referred to as a surgical tool, can be a part of a system for performing a medical procedure, such as a system including a robotic probe and/or a robotically controlled tool. Surgical instrument 100′ can include similar components and otherwise be of similar construction and arrangement to instrument 100 described hereabove in reference to FIG. 1A. As shown in FIG. 1B, one of the controls of handle 105 can include an articulation mechanism 140 that serves as a spring-loaded trigger with respect to the end effector 150. Activation of the spring-loaded trigger (e.g. a user squeezes the spring-loaded trigger with multiple fingers) induces a movement and/or an actuation of end effector 150. Steering mechanism 130 can include a ball and socket type interface between shaft 110 and handle 105, as described in applicant's co-pending U.S. patent application Ser. No. 14/402,224, filed Nov. 19, 2014, the contents of which are incorporated herein by reference for all purposes.

As described herein, tools 100 can be constructed and arranged to be slidingly received within a lumen, such as a working channel of a robotic introducer system, or other lumen configured to guide tools 100 to a surgical site. End effectors 150, articulation regions 120, and/or at least a portion of shafts 110 (e.g. flexible portion 115), can each be constructed and arranged to allow tool 100 to be slidingly received within a lumen with an inner diameter of less than 6 mm, such as less than 5 mm, less than 4 mm, less than 3 mm, or less than 2 mm.

Referring now to FIG. 1C, a side perspective view of another embodiment of a surgical instrument is illustrated, consistent with the present inventive concepts. Surgical instrument 1001, also referred to as a surgical tool, can be a part of a system for performing a medical procedure, such as a system including a robotic probe and/or a robotic tool controller. Surgical instrument 1001 can include similar components and otherwise be of similar construction and arrangement to instruments 100 and 100′ described hereabove in reference to FIGS. 1A and 1B. As shown in FIG. 1C, surgical instrument 1001 can comprise shaft 110. Shaft 110 can comprise a flexible shaft, and the distal end of shaft 110 can be directly or indirectly coupled to the proximal end of an articulation region 120. Shaft 110 can extend along a same axial direction as articulation region 120, or a different direction, for example, during movement of articulation region 120. An end effector 150 can be coupled to the distal end of articulation region 120, as described herein.

The proximal end of shaft 110 can be coupled to a control assembly 1410, which is operably attached to articulation region 120 and/or end effector 150 via one or more linkages 1445 extending through shaft 110. Control assembly 1410 can comprise an interface assembly 4100, an outer rotating assembly 4200, and an inner rotating assembly 4300. In some embodiments, outer rotating assembly 4200 further comprises a support assembly 4210 extending distally therefrom. Support assembly 4210 can be operably attached to the proximal end of shaft 110. Control assembly 1410 can be configured to manipulate the proximal portion of linkage(s) 1445, controlling the articulation and/or actuation of articulation region 120 and/or end effector 150, respectively. Surgical instrument 1001 can be of similar construction and arrangement to the similar components described in applicant's co-pending U.S. Patent Application Ser. No. 62/614,225, filed Jan. 5, 2018 the contents of which are incorporated herein by reference for all purposes. Surgical instrument 1001 can operably attach to a robotic controller, such as a robotic controller described in applicant's co-pending U.S. Patent Application Ser. No. 62/614,228, filed Jan. 5, 2018 the contents of which are incorporated herein by reference for all purposes.

In some embodiments, surgical instrument 1001 comprises a functional element, for example functional element 199 positioned proximate the distal end of instrument 1001. In some embodiments, functional element 199 comprises an identifier, such as an RFID, configured to indicate to a robotic controller one or more properties of instrument 1001 (e.g. the type of end effector 150 attached to instrument 1001). In some embodiments, functional element 199 comprises an functional element selected from the group consisting of: a visualization element, such as camera; a visualizable element, for example a radiopaque element for visualization under X-ray or fluoroscopy; a temperature sensor; a pH sensor; a conductive element, for example an electrode configured to pair with a conductive end effector 150 for bipolar energy delivery; a transducer, such as an ultrasound or an audible transducer; an environmental sensor; and combinations of one or more of these.

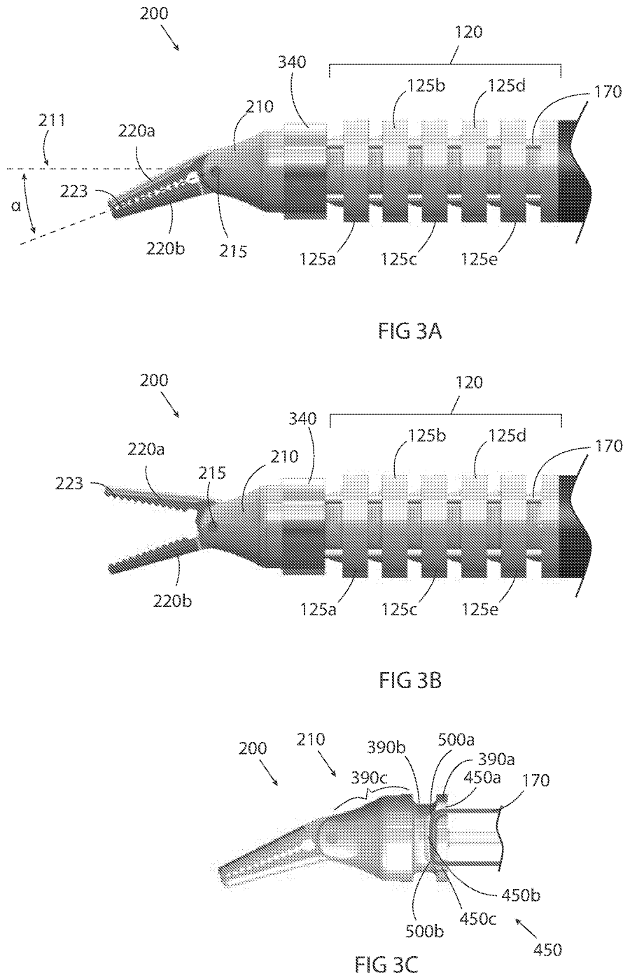

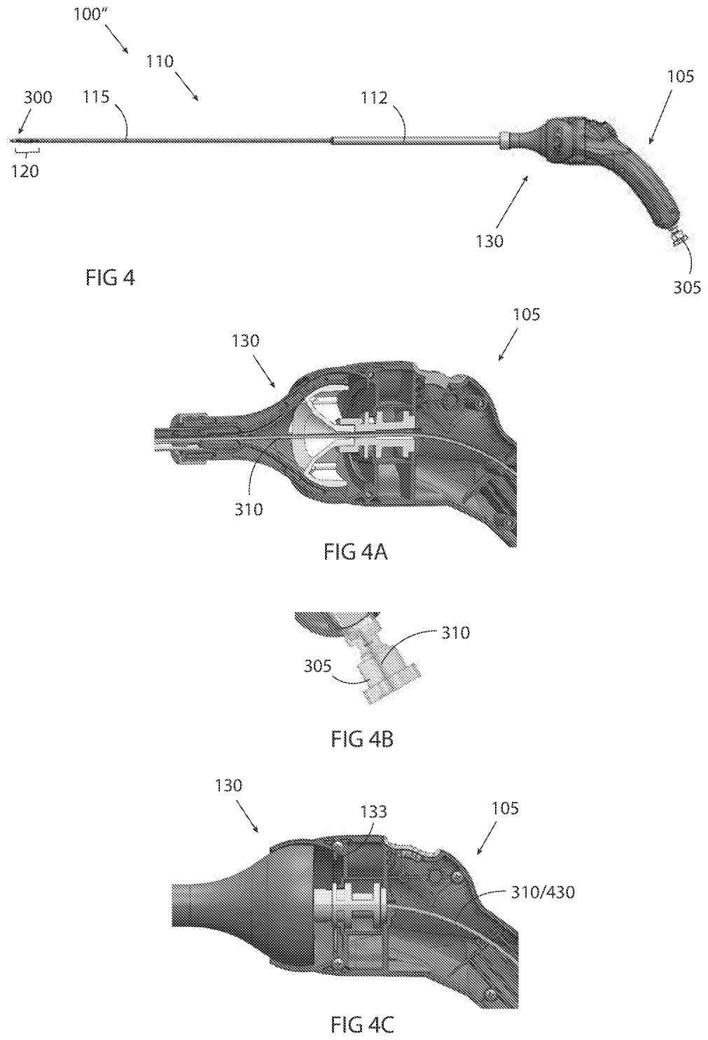

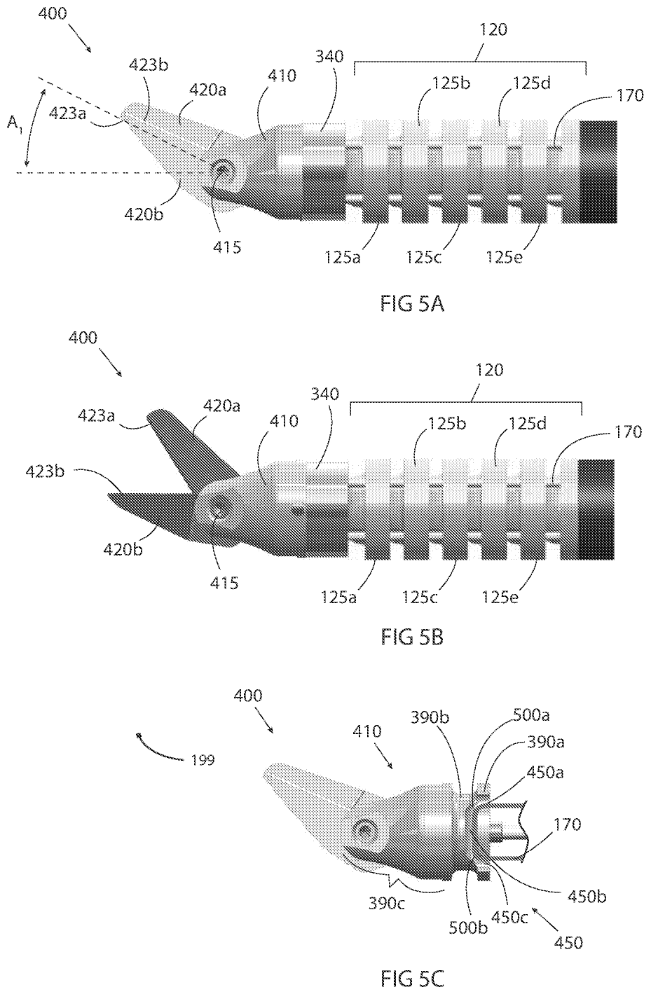

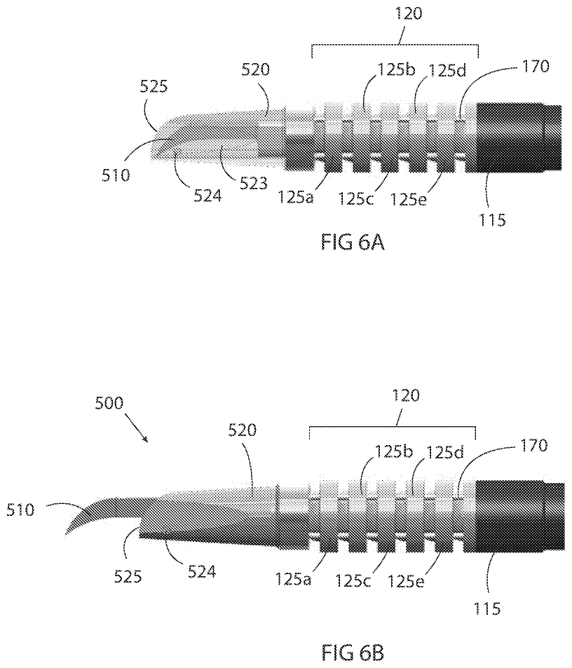

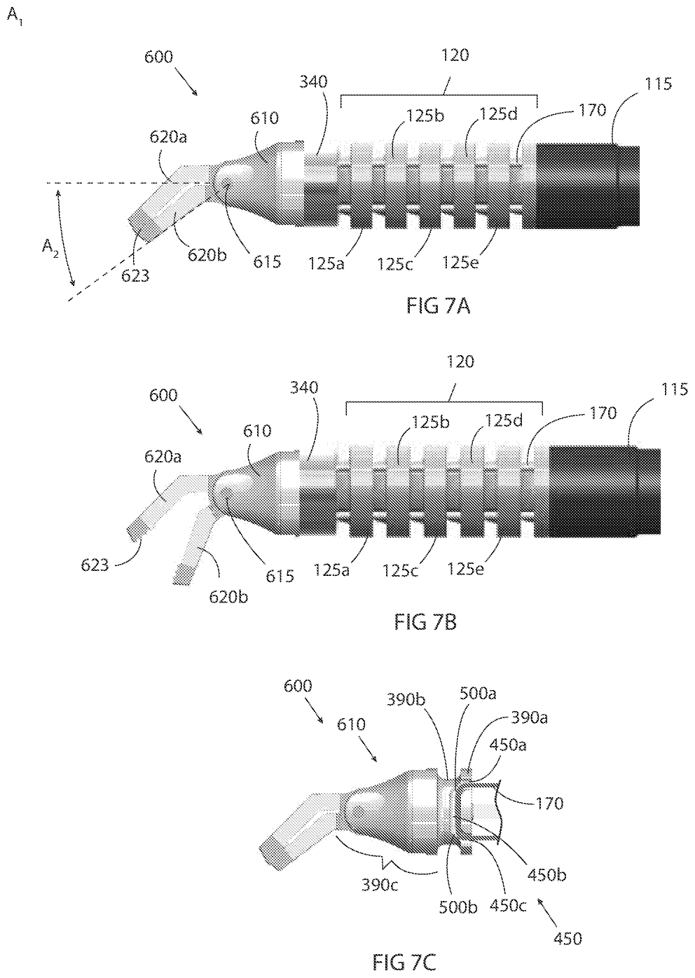

Referring now to FIGS. 2A-E, perspective views of various embodiments of an end effector for a surgical instrument are illustrated, consistent with the present inventive concepts. As shown in FIG. 2A and as described herebelow in reference to FIGS. 3A-C, an end effector 200 can comprise a grasper. The jaws of end effector 200 can comprise a thickness of less than 1 mm and can comprise a length of less than 5 mm. The clevis of end effector 200 can comprise a length of less than 4 mm, such as less than 3.5 mm. As shown in FIG. 2B and as described herebelow in reference to FIGS. 4 and 4A-C, an end effector 300 can comprise a light-delivery element (e.g. an element configured to deliver laser-based light energy). The shaft of end effector 300 can comprise a length of less than 4 mm, such as less than 3.5 mm. As shown in FIG. 2C and as described herebelow in reference to FIGS. 5A-C, an end effector 400 can comprise a dual-bladed cutting tool, such as scissors. The blades of end effector 400 can comprise a width of less than 1 mm and can comprise a length of less than 8 mm. The clevis of end effector 400 can comprise a length of less than 4 mm, such as less than 3.5 mm. As shown in FIG. 2D and as described herebelow in reference to FIGS. 6A-B, an end effector 500 can comprise a single bladed cutting tool, such as a knife. The blade of end effector 500 can comprise a width of less than 1 mm, such as less than 0.5 mm, and a can comprise a length of less than 10 mm. The housing of end effector 500 can comprise a length of less than 4 mm, such as less than 3.5 mm. As shown in FIG. 2E and as described herebelow in reference to FIGS. 7A-C, an end effector 600 can comprise forceps. The jaws of end effector 600 can comprise a depth of less than 3 mm, such as less than 2 mm, and can comprise a length of less than 3 mm, such as less than 2 mm. The clevis of end effector 600 can comprise a length of less than 4 mm, such as less than 3.5 mm.

Referring now to FIGS. 3A-B, side perspective views of a grasper end effector in a closed and open position, respectively, for a surgical instrument are illustrated, consistent with the present inventive concepts. End effector 200 can comprise a grasper with a clevis 210 that includes a hinge point 215. At least one element of the end effector can pivot about the hinge point 215. A proximal end of clevis 210 can be coupled to a distal end of articulation region 120. In some embodiments, articulation region 120 comprises a plurality of articulating links 125, links 125a-e shown. In some embodiments, the articulating links 125 include concave surfaces that mate with convex surfaces of neighboring links. A first jaw 220a and a second jaw 220b can both extend from the distal end of clevis 210. First jaw 220a and second jaw 220b can transition between a closed position (as shown in FIG. 3A) and an open position (as shown in FIG. 3B) via hinge point 215. As shown in FIG. 3A, while in the closed position, first jaw 220a and second jaw 220b can comprise a 20° biased angle α relative to a median line 211 of clevis 210. In some embodiments, biased angle α comprises an angle of approximately 22°. The biased angle allows for greater maneuverability and a farther reach of surgical instrument 100, as may be necessary inside the tight constraints of the anatomy of the patient. In some embodiments, the diameter of the articulating links 125 is greater than the diameter of the clevis 210. A distal ring 340 is positioned to cover a proximal portion of the clevis 210.

Jaws 220a,b can comprise teeth-like projections, projections 223, configured to engage and grasp tissue within the patient. In some embodiments, first jaw 220a and second jaw 220b are constructed and arranged to have a cambered design that biases jaws 220a,b such that their distal ends (tips) make contact with each other prior to the more proximal portions of jaws 220a,b touching, when transitioning from the open position to the closed position. When gripping tissue, the cambered design allows for jaws 220a,b to be positioned relatively parallel to each other, resulting in an increase of surface area between the two jaws that can provide increased tissue retention force.

Referring now to FIG. 3C, a side view of a grasper end effector, with the distal ring 340 removed. The clevis 210 can include a proximal region 390a, a neck region 390b, and a distal region 390c. The maximum outer diameter of the neck region 390b can be different than, for example less than, the maximum outer diameter of the proximal region 390a and the maximum outer diameter of the distal region 390c.

A movement of handle 105 can provide tension or slack to one or more steering cables 170, thereby adjusting an articulation state of articulation region 120. An articulation of handle 105 configured with a single degree of freedom can cause articulation region 120 to move in a single plane along a single pathway of motion. An articulation of handle 105 configured with two degrees of freedom can permit articulation region 120 to be manipulated to reach anywhere on a surface of at least a partial sphere.

In some embodiments, the articulation region 120 can be manipulated with four steering cables 170.

The clevis 210 can include at least one cable channel 450. A steering cable 170 can be positioned in the cable channel 450. The cable channel 450 can include a first end 450a, a second end 450c and a middle region 450b between the first and second ends 450a, 450c.

In some embodiments, adjacent regions of the cable channel 450 can be joined at one or more pinch regions 500, pinch regions 500a,b shown. Each pinch region 500a,b is designed to prevent the tension applied to the cable in one region from being transferred to the same cable in an adjacent region. In some embodiments, this can be achieved by bonding the cable to the cable channel 450 at the pinch region 500. In other embodiments, the diameter of the cable channel 450 at the pinch region 500 can be less than the diameter of the cable channel at other locations, thereby securing the cable in place and preventing the tension applied to the cable in one region from being transferred to the same cable in the adjacent region. In some embodiments, the clevis 210 is unitary and formed from a single piece of material.

Referring now to FIG. 4, a side perspective view of a surgical instrument with a light-delivering end effector is illustrated, consistent with the present invention. FIGS. 4A-C illustrate views of portions of a handle of a surgical instrument, also consistent with the present inventive concepts. Surgical instrument 100″ can be configured to be flexible and/or steerable (e.g. via a manual control of instrument 100″ and/or via robotic control), such as to support use within and/or alongside robotic surgical devices. Surgical instrument 100″ can include similar components and otherwise be of similar construction and arrangement to instrument 100 and/or instrument 100′ described hereabove in reference to FIGS. 1A and 1B, respectively. Surgical instrument 100″ can include a handle 105, a steering mechanism 130, and an articulation region 120. Surgical instrument 100″ can include a surgical tool shaft, shaft 110, comprising a rigid portion 112 and a flexible portion 115. A distal end of shaft rigid portion 112 can be coupled to a proximal end of shaft flexible portion 115. A distal end of shaft 110 can be directly or indirectly coupled to articulation region 120. Shaft rigid portion 112 and shaft 110 can extend along a same axial direction as articulation region 120, or a different direction, for example, during movement of articulation region 120. Articulation region 120 can comprise one or more articulating links, links 125 (not shown). A distal end of articulation region 120 can include an end effector 300 comprising a light-delivering element. In some embodiments, the proximal end of handle 105 includes a connector 305.

As shown in FIG. 4A, a side sectional view of a portion of handle 105, a lumen for a light-delivering element, lumen 310, extends between connector 305 and end effector 300 (e.g. lumen 310 runs continuously throughout the entire surgical instrument 100″). As shown in FIG. 4B, a side view of a proximal portion of handle 105, a proximal portion of lumen 310 can terminate within connector 305 positioned on the proximal end of handle 105. In some embodiments, lumen 310 includes one or more slits to allow for a Tuohy Burst valve to secure lumen 310 to connector 305. As shown in FIG. 4C, a partial cutaway side view of a portion of handle 105, a proximal portion of steering mechanism 130 can include one or more articulation limiting elements 133. The articulation limiting elements 133 are configured to limit the motion of surgical instrument 100″ in order to prevent breakage of light-delivery element fibers, optical fibers 430, positioned within lumen 310. In the embodiment shown in FIG. 4A, the articulation limiting element is in the form of a ring, but in other embodiments the articulation limiting elements 133 may take different forms. For example, in some embodiments, the articulation limiting elements 133 can be one or more blocks or tabs that do not connect to faun a complete ring. In other embodiments, the articulation limiting elements 133 can include friction regions on the inner surface of the surgical instrument to limit manipulation of the handle with respect to the steering mechanism.

In some embodiments, the articulation limiting elements 133 can be configured to provide variable resistance to the motion of the surgical instrument 100″. For example, as the user manipulates the handle into a position that is likely to damage optical fibers 430, the degree of friction between the handle and the steering mechanism can be configured to gradually increase.

Referring now to FIGS. 5A-B, side perspective views of a scissors-based end effector is illustrated, shown in a closed position and in an open position, respectively, and consistent with the present inventive concepts. End effector 400 can comprise a scissors with a clevis 410 that includes a hinge point 415. A proximal end of clevis 410 can be coupled to a distal end of articulation region 120. In some embodiments, articulation region 120 comprises multiple articulating links 125, links 125a-e shown. A distal end of clevis 410 can include a first jaw 420a (e.g. with a sharp blade edge) and a second jaw 420b (e.g. with a mating, sharp blade edge), both constructed and arranged to extend from the distal end of clevis 410. First jaw 420a and second jaw 420b can transition between a closed position (as shown in FIG. 5A) and an open position (as shown in FIG. 5B) via hinge point 415. As shown in FIG. 5A, while in the closed position, first jaw 420a and second jaw 420b can comprise a biased angle, angle A₁, such as an approximately 30° biased angle relative to a medial line of clevis 410. The magnitude of angle A₁ allows for greater maneuverability and a further reach of surgical instrument 100 inside the tight constraints of the anatomy of the patient. In some embodiments, angle A₁ comprises a biased angle of approximately 45°, such as approximately 43°. A distal ring 340 is positioned to cover a proximal portion of the clevis 410.

In some embodiments, first jaw 420a can comprise a blade with a blunt distal end and second jaw 420b comprises a blade with a sharp distal end. First jaw 420a can comprise a length that is greater than second jaw 420b. As shown in FIG. 5A, when in the closed position, inner edges 423a,b of jaws 420a,b, respectively, overlap such that the blunt distal end of first jaw 420a extends beyond the sharp distal end of second jaw 420b (e.g. the blunt distal end covers or otherwise protects the sharp distal end when in the closed position). The overlapping configuration of jaws 420a,b in the closed position allows for ease of insertion of end effector 400 through working channels or other delivery lumens (“working channels” herein) of an introducer (e.g. a robotic surgical introducer) and protects jaws 420a,b from damage during insertion, and protects the working channels from damage during the insertion.

As shown in FIG. 5B, while in the open position, the sharp distal end of second jaw 420b can be constructed and arranged to relatively align and extend along a same axial direction as clevis 410, aiding in the dissection of a tissue in the patient.

Referring now to FIG. 5C, a side view of a scissors-based end effector, with the distal ring 340 removed. In some embodiments, the clevis 410 can include a proximal region 390a, a neck region 390b, and a distal region 390c. The maximum outer diameter of the neck region 390b can be different than, for example less than, the maximum outer diameter of the proximal region 390a and the maximum outer diameter of the distal region 390c.

In some embodiments, the articulation region 120 can be manipulated with four steering cables 170.

The clevis 410 can include at least one cable channel 450. A steering cable 170 can be positioned in the cable channel 450. The cable channel 450 can include a first end 450a, a second end 450c and a middle region 450b between the first and second ends 450a, 450c.

In some embodiments, adjacent regions of the cable channel 450 can be joined at one or more pinch regions 500, pinch regions 500a,b shown. Each pinch region 500a,b is designed to prevent the tension applied to the cable in one region from being transferred to the same cable in an adjacent region. In some embodiments, this can be achieved by bonding the cable to the cable channel 450 at the pinch region 500. In other embodiments, the diameter of the cable channel 450 at the pinch region 500 can be less than the diameter of the cable channel at other locations, thereby securing the cable in place and preventing the tension applied to the cable in one region from being transferred to the same cable in the adjacent region. In some embodiments, the clevis 410 is unitary and formed from a single piece of material.

Referring now to FIGS. 6A-B, a partial cutaway side view of a knife-based end effector in a retracted position and a side perspective view of the end effector in an extended position are illustrated, respectively, consistent with the present inventive concepts. End effector 500 can comprise a blade 510 configured to retract within, and extend from, a housing 520. Blade 510 can comprise a sharp distal end including a curve or other smoothly rounded bend (e.g. blade 510 shown in FIGS. 6A-B comprises a sickle shape). Housing 520 can include a recess 523 with a motion-limiting element, projection 524, and a distal end opening 525. A proximal end of housing 520 can be coupled to a distal end of articulation region 120. In some embodiments, articulation region 120 comprises multiple articulating links 125, links 125a-e shown.

As shown in FIG. 6A, blade 510 can be retracted into recess 523 via opening 525, such that blade 510 is sufficiently surrounded by housing 520 (e.g. the sharp distal end of blade 510 does not extend beyond opening 525). In some embodiments, projection 524 is positioned to engage blade 510 to limit its retraction (e.g. to prevent it from over-retracting into recess 523). Retraction of blade 510 into recess 523 allows for ease of insertion of end effector 500 through working channels of a robotic or other introducer device, protects blade 510 from damage during insertion, and protects the working channels from damage during the insertion. As shown in FIG. 6B, blade 510 can be extended out of recess 523 via opening 525, such that blade 510 exits housing 520 (e.g. the sharp distal end of blade 510 extends beyond opening 525).

Referring now to FIGS. 7A-B, a side perspective view of a forceps-based end effector in a closed position and a side perspective view of the end effector in an open position are illustrated, respectively, consistent with the present inventive concepts. End effector 600 can comprise forceps with a clevis 610 that includes a hinge point 615. A proximal end of clevis 610 can be coupled to a distal end of articulation region 120. In some embodiments, articulation region 120 comprises multiple articulating links 125, links 125a-e shown. A distal end of clevis 610 can include a first jaw 620a and a second jaw 620b, both constructed and arranged to extend from the distal end of clevis 610. First jaw 620a and second jaw 620b can transition between a closed position (as shown in FIG. 7A) and an open position (as shown in FIG. 7B) via hinge point 615. As shown in FIG. 7A, while in the closed position, first jaw 620a and second jaw 620b can comprise a bias angle, angle A₂, such as an approximately 40° biased angle relative to a median line of clevis 610. The magnitude of angle A₂ allows for greater maneuverability and a farther reach of surgical instrument 100 inside the tight constraints of the anatomy of the patient. In some embodiments, angle A₂ comprises a biased angle of approximately 45°, such as approximately 43°. A distal ring 340 is positioned to cover a proximal portion of the clevis 610.

Jaws 620a,b can comprise teeth-like projections, projections 623, configured to engage and grasp a tissue within the patient. In some embodiments, first jaw 620a and second jaw 620b are constructed and arranged to have a cambered design that biases the tips of jaws 620a,b to touch first when transitioning from the open position to the closed position. When gripping tissue, the cambered design allows for jaws 620a,b to lay more parallel to each other, resulting in an increase of surface area between the two jaws for greater tissue retention.

In some embodiments, jaws 620a,b can each comprise an opening 625, openings 625a,b, respectively. These elements are shown in FIG. 2E. Openings 625a,b can be constructed and arranged to receive tissue when jaws 620a,b are in the closed position. Openings 625a,b can comprise a length of less than 2 mm and a width of less than 1.75 mm.

Referring now to FIG. 7C, a side view of a forceps-based end effector, with the distal ring 340 removed is illustrated. The clevis 610 can include a proximal region 390a, a neck region 390b, and a distal region 390c. The maximum outer diameter of the neck region 390b can be different than, for example less than, the maximum outer diameter of the proximal region 390a and the maximum outer diameter of the distal region 390c.

A movement of handle 105 can provide tension or slack to one or more steering cables 170, thereby adjusting an articulation state of articulation region 120. An articulation of handle 105 configured with a single degree of freedom can cause articulation region 120 to move in a single plane along a single pathway of motion. An articulation of handle 105 configured with two degrees of freedom can permit articulation region 120 to be manipulated to reach anywhere on a surface of at least a partial sphere.

In some embodiments, the articulation region 120 can be manipulated with four steering cables 170.

The clevis 610 can include at least one cable channel 450. A steering cable 170 can be positioned in the cable channel 450. The cable channel 450 can include a first end 450a, a second end 450c and a middle region 450b between the first and second ends 450a, 450c.

In some embodiments, adjacent regions of the cable channel 450 can be joined at one or more pinch regions 500, pinch regions 500a,b shown. Each pinch region 500a,b is designed to prevent the tension applied to the cable in one region from being transferred to the same cable in an adjacent region. In some embodiments, this can be achieved by bonding the cable to the cable channel 450 at the pinch region 500. In other embodiments, the diameter of the cable channel 450 at the pinch region 500 can be less than the diameter of the cable channel at other locations, thereby securing the cable in place and preventing the tension applied to the cable in one region from being transferred to the same cable in the adjacent region. In some embodiments, the clevis 610 is unitary and formed from a single piece of material.

The above-described tools are described herein in relation to the articulation region 120 of surgical instrument 100, surgical instrument 100′, and surgical instrument 100″. In some embodiments, the herein-described tools can be connected to a non-articulating surgical instrument (i.e. a surgical instrument that does not have an articulation region).

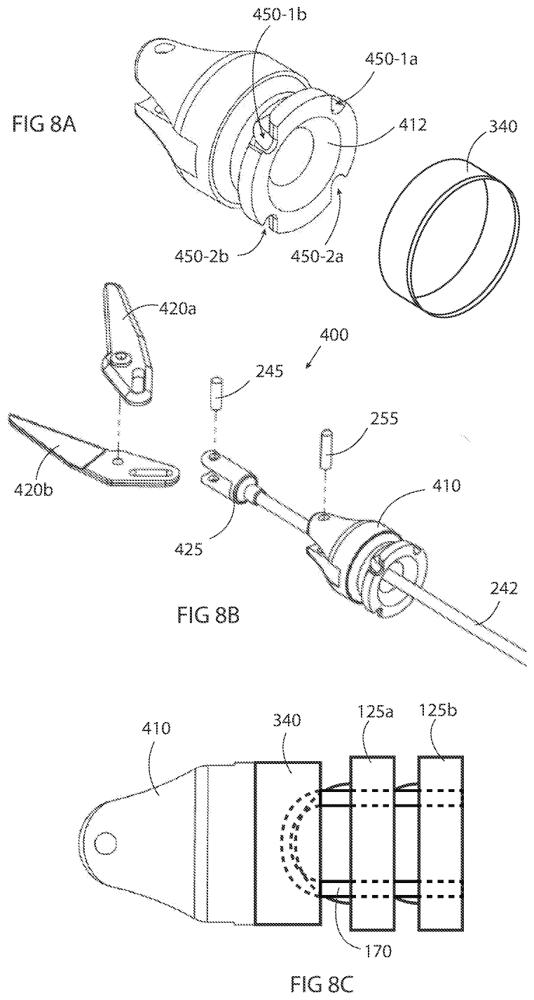

Referring now to FIG. 8A, a perspective view of an embodiment of the clevis 410 and the distal ring 340 is illustrated, consistent with the present inventive concepts. In this embodiment, a concave surface 412 of the proximal end of the clevis 410 can be seen. As described herein, the concave surface 412 of the clevis 410 interfaces with a corresponding convex surface of a distal-most articulating link 125a of the articulation region 120 of the surgical instrument 100.

Referring now to FIG. 8B, a perspective exploded view of an embodiment of the clevis 410, the end effector 400, and an actuating cable 242, is illustrated, consistent with the present inventive concepts. In some embodiments, the clevis 410 comprises a first jaw 420a, a second jaw 420b, and a jaw coupler 425. A scissors pin 245 couples the first jaw 420a, the second jaw 420b, and the jaw coupler 425. In some embodiments, a clevis pin 255, couples the first jaw 420a, the second jaw 420b, and the clevis 410. In some embodiments, the jaw coupler 425 is connected to the distal end of actuating cable 242. The actuating cable 242 allows the user to operate the end effector 400 from the proximal end of the surgical instrument 100 at the handle 105.

Referring now to FIG. 8C, a side view of an embodiment of the clevis 410, steering cable 170, articulating links 125a,b, and distal ring 340. The clevis 410 includes a concave surface that mates with a convex surface of the neighboring articulating link 125a. The distal ring 340 covers the proximal region 390a of the clevis 410 as described herein.

Referring now to FIGS. 9A-C, side views of an adaptor and two detachable end effectors are illustrated, respectively, consistent with the present inventive concepts. Tool 100 can comprise an adaptor 750, for removably attaching an end effector 700 to articulation region 120 of tool 100. Adaptor 750 can comprise an extension, neck 751, extending from a base 755. Base 755 operably attaches to articulation region 120, for example to a distal link 125. In some embodiments, distal link 125 comprises base 755, and neck 751 extends therefrom. Neck 751 can include a radial projection, barb 752. Barb 752 can engage a portion of a connected end effector 700, as described herebelow, to help prevent unwanted detachment of the end effector 700 from adaptor 750. Neck 751 can further include a connector 753, operably attached to a conduit 754.

As shown in FIG. 9B, end effector 700a can comprise a cutter, blade 705. As shown in FIG. 9C, end effector 700b can comprise a scissor and/or grasper, jaws 715. End effectors 700a and 700b are referred to collectively as end effector 700 herein. In some embodiments, end effectors 700 can comprise an active device, such as ultrasonic and/or RF devices, such as an ultrasonic cutter and/or a bipolar RF grasper. In these embodiments, as shown, end effector 700 can comprise a transducer 725. End effector 700 can comprise a housing 720, surrounding a recess 723 for slidingly receiving neck 751 of adaptor 750, operably attaching end effector 700 thereto. End effector 700 can comprise a connector 724, configured to operably attach to connector 753 of adaptor 750 when neck 751 is inserted into recess 723. Connectors 753 and 724 can operably connect conduit 754 to a conduit 726 of end effector 700. Conduit 726 is operably attached to transducer 725, positioned within housing 720. Conduit 754 can extend through tool 100, and operably attach to a power or other energy source, such as a power source operably attached to handle 105 of tool 100. Transducer 725 can generate, transform, or otherwise transmit power and/or energy to blade 705 and/or jaws 715. For example, transducer 725 can receive electrical energy via conduit 754, and provide ultrasonic energy to blade 705 and/or jaws 715.

Blade 705 can extend from housing 720. In some embodiments, blade 705 can be of similar construction and arrangement to blade 510 of FIGS. 6A-B. Jaws 715 can extend from housing 720 and be articulatable, such as via one or more control cables not shown, but similar to those described hereabove. In some embodiments, jaws 715 can be of similar construction and arrangement to jaws 220a,b, jaws 420a,b, and/or jaws 620a,b, described herein.

In some embodiments, housing 720 can comprise a hook 721. A filament, leash 722, can be secured to hook 721, such as to allow for retrieval and/or placement of end effector 700 from and/or into a surgical site, as described herebelow.

As described herein, tools 100 can be constructed and arranged to be slidingly received within a lumen, such as a working channel of a robotic introducer system, or other lumen configured to guide tools 100 to a surgical site. Adaptor 750, articulation regions 120, and/or at least a portion of shafts 110 (e.g. flexible portion 115), can each be constructed and arranged to allow tool 100 to be slidingly received within a lumen with an inner diameter of less than 6 mm, such as less than 5 mm, less than 4 mm, less than 3 mm, or less than 2 mm. In some embodiments, end effectors 700a,b are too large to be slidingly received within the lumen, and are constructed and arranged to be operably attached to adaptor 750 after tool 100 has been received by the lumen, and exited the distal end thereof. In some embodiments, end effectors 700a,b comprise a diameter greater than the diameter of the lumen. Additionally or alternatively, end effectors 700a,b can comprise a rigid length greater than can be slidingly received within the lumen, for example when the lumen is in a tortuous geometry.

In some embodiments, a robotic introducer system can be advanced to a surgical site, for example a transabdominal surgical site, through an introducer such as a trocar, positioned through the skin of the patient. A tool 100, with adaptor 750 attached to the distal end of articulation region 120, can be advanced through a working channel of the robotic introducer system to the surgical site. A second tool, for example tool 600 described herein, can also be advanced through the introducer to the surgical site. An end effector 700 can subsequently (or previously) be placed in or near the surgical site, such as through an auxiliary port of a trocar. A user can maintain control of end effector 700 via leash 722. In some embodiments, adaptor 750 can then be steered (e.g. manually or robotically) towards end effector 700. In some embodiments, adaptor 750 can attach to end effector 700 via a press fit, or other action that does not require additional tools. In some embodiments, tool 600 can grasp end effector 700, and assist in the connection between end effector 700 and adaptor 750. In some embodiments adaptor 750 and/or end effector 700 can comprise magnets or other elements configured to assist in the attachment. In some embodiments, tool 100 can be retracted into the introducer, and end effector 700 can disconnect from adaptor 750, such that tool 100 can be retracted, and end effector 700 can be retrieved from the surgical site via leash 722. In some embodiments, multiple end effectors, for example end effectors 700a and 700b, can be introduced to the surgical site, and tool 100 can connect and disconnect from either during a procedure, based on the needs of the user. In some embodiments, each end effector 700 can comprise a functional element 199, as described hereabove in reference to FIG. 1C. Functional element 199 can comprise an identifier, for example and RFID, configured to indicate to a tool and/or a tool controller the properties of the attached end effector 700.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A surgical instrument comprising:
a proximal end;
a distal end having an articulation region, a movement of which is controlled from the proximal end; and
a shaft between the proximal end and the distal end;
a plurality of steering cables extending from the proximal end to the distal end; and
an end effector comprising a clevis, the clevis including at least one channel that receives a portion of a steering cable of the plurality of steering cables.

2.-58. (canceled)
